(19) United States
(12) Patent Application Publication
(10) Pub. No.: US 2016/0340280 A1
(43) Pub. Date: Nov. 24, 2016

(54) PROCESS FOR PREPARING 2,2'-DIHYDROXY-3,3'-DI-TERT-BUTYL-5,5'-DIMETHOXY-1,1'-BIPHENOL

(71) Applicant: EVONIK DEGUSSA GMBH, Essen (DE)

(72) Inventors: Katrin Marie DYBALLA, Recklinghausen (DE); Andrea CHRISTIANSEN, Rostock (DE); Robert FRANKE, Marl (DE); Dirk FRIDAG, Haltern am See (DE); Burkard KREIDLER, Essen (DE)

(73) Assignee: EVONIK DEGUSSA GMBH, Essen (DE)

(21) Appl. No.: 15/157,962

(22) Filed: May 18, 2016

(30) Foreign Application Priority Data
May 20, 2015 (EP) 15168364

(51) Int. Cl. C07C 41/30; C07C 41/36

(52) U.S. Cl. CPC C07C 41/30; C07C 41/36

(57) ABSTRACT

Process for preparing 2,2′-dihydroxy-3,3′-di-tert-butyl-5,5′-dimethoxy-1,1′-biphenol.

### PROCESS FOR PREPARING 2,2′-DIHYDROXY-3,3′-DI-TERT-BUTYL-5,5′-DIMETHOXY-1,1′-BIPHENOL

The present invention relates to a novel process for preparing 2,2′-dihydroxy-3,3′-di-tert-butyl-5,5′-dimethoxy-1,1′-biphenol by oxidative coupling of 3-tert-butyl-4-hydroxyanisole (3-BHA).

Symmetric biphenols are great interest for industrial applications (cf. WO 2005/042547). These are employed particularly as ligand components for catalysts. In this case, the biphenol can be used, for example, as ligand unit in enantioselective catalysis (cf. Y. Chen, S. Yekta, A. K. Yudin, Chem. Rev. 2003, 103, 3155-3211; J. M. Brunel Chem. Rev. 2005, 105, 857-898; S. Kobayashi, Y. Mori, J. S. Fossey, Chem. Rev. 2011, 11, 2626-2704).

However, the direct coupling of phenols to give the corresponding biphenol derivatives continues to be a challenge since these reactions are often neither regio- nor chemoselective.

The literature describes a multitude of potential oxidizing agents capable of coupling phenols, especially o,p-disubstituted phenols, to give 2,2′-dihydroxybiphenols. In this context, particularly potassium hexacyanoferrate(III), K₃[Fe(CN)₆ (see: a) Adv. Synth. Catal. 2004, 346, 993-1003; b) J. Org. Chem. 2011, 76, 8376-8385) and sodium peroxodisulphate play a crucial role.

However, the two aforementioned oxidizing agents are relatively costly, which has an immediate effect on the overall cost of the product in an industrial scale synthesis. Moreover, in the case of use of potassium hexacyanoferrate(III) and in the case of use of sodium peroxodisulphate, unwanted salts and, in the former case, possibly even toxic cyanides occur as by-products. Thus, a multitude of by-products are formed, the removal of which from the desired target product and the disposal of which is in need of improvement from an ecological and economic point of view.

Alternatively, the biphenols can also be prepared electrochemically (see: M. Malkowsky, U. Griesbach, H. Pütter, S. R. Waldvogel, Novel Template-directed Anodic Phenol Coupling Reaction, Chem. Eur. J. 2006, 12, 7482-7488). In the process described therein, BHA-boronic acid is converted electrochemically in acetonitrile, in order to obtain the desired biphenol. This process thus requires a BHA-borate, which causes an additional synthesis step and unnecessary borate wastes.

A further process uses inexpensive atmospheric oxygen. The reaction proceeds in aqueous NaOH, while air is blown into the mixture at 80° C. (see: a) US 4.717.775 (UCC); b) J. Organomet. Chem. 1994, 471, 201.; c) J. Org. Chem. 1989, 54, 41154217). When working with atmospheric oxygen or optionally pure oxygen in industrial scale production, additional precautionary measures have to be observed, which generally also makes the process more expensive again.

Another reaction that has been described is that using copper(II), e.g. copper chloride/TMEDA (J. Mol. Cat., 1983, 83, 17). The reaction is conducted in methanol without addition of base. The reaction can be conducted at room temperature, but with very long reaction times. A disadvantage here is the use of metal reagents which could in some cases be troublesome in the product (for example in the case of further reactions).

Hydrogen peroxide is the second least expensive oxidizing agent (after atmospheric oxygen) and reacts much more quickly with BHA than air. Normally, the metered addition of H₂O₂ takes one to two hours and, after a further 30 minutes, the reaction has ended. The reaction proceeds in aqueous NaOH using sodium laurylsulphate (sodium dodecylsulphate) as an inexpensive and efficient phase transfer agent. This reaction was described in 1981 by Ciba (cf. EP 35965 B1). The aforementioned European patent specification discloses a process for preparing 2,2′-dihydroxybiphenyl compounds, wherein the reactant used, in contrast to the present invention, is not 3-tert-butyl-4-hydroxyanisole (3-BHA). Instead, in EP 35965 B1, only phenols of the general formula

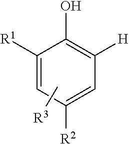

having, in the para position to the phenolic hydroxyl group, hydrogen, C₁-C₁₈-alkyl, C₂-C₆-alkenyl, C₅-C₇-cycloalkyl optionally substituted by C₁-C₄-alkyl radicals, phenyl or C₇-C₈-phenylalkyl as substituents R² are used as reactant.

Only in the case that the R¹ and R³ substituents together are a butadi-1,3-enyl-1,4-ene radical bonded to the 3,4 or 3′,4′ positions can R² be a -(CH₂)ₙCOOR⁴ group having heteroatoms, where R⁴ is C₁-C₁₈-alkyl and n =0, 1 or 2. In all other cases, without exception, hydrocarbons are described as possible substituents for R², and no pointer is given that these radicals could be substitutable by a heteroatom. Especially in the case in which R¹ is an alkyl radical, for example a tert-butyl radical, possible substituents are specified for R² are exclusively hydrocarbons.

The problem addressed by the present invention was thus that of providing a process for preparing 2,2′-dihydroxy-3,3′-di-tert-butyl-5,5′-dimethoxy-1,1′-biphenol which does not have the disadvantages described in connection with the prior art. A preferred problem was additionally that of selectively preparing 2,2′-dihydroxy-3,3′-di-tert-butyl-5,5′-dimethoxy-1,1′-biphenol, in which a minimum amount of by-products occurs.

This object is achieved by a process according to claim 1.

Process for preparing 2,2′-dihydroxy-3,3′-di-tert-butyl-5,5′-dimethoxy-1,1′-biphenol by oxidative coupling of 3-tert-butyl-4-hydroxyanisole, comprising the process steps of:
a) heating a mixture comprising at least one solvent, an inorganic base and 3-tert-butyl-4-hydroxyanisole to a temperature above room temperature,
b) adding a hydrogen peroxide solution to the mixture,
c) removing the 2,2′-dihydroxy-3,3′-di-tert-butyl-5,5′-dimethoxy-1,1′-biphenol product.

The oxidative coupling of 3-tert-butyl-4-hydroxyanisole (3-BHA) to give 2,2′-dihydroxy-3,3′-di-tert-butyl-5,5′-dimethoxy-1,1′-biphenol (1) proceeds according to equation 1 (Eq. 1):

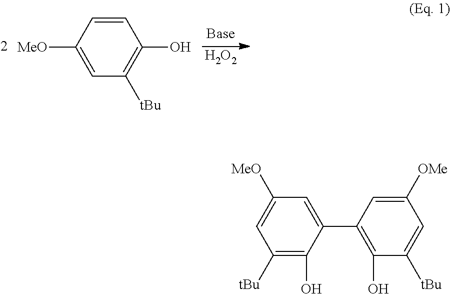

Since the 3-tert-butyl-4-hydroxyanisole (3-BHA) reactant is an o,p-disubstituted phenol, only a sterically unhindered arrangement leads to recombination of two reactant molecules, which, in most cases, leads to the homo-coupling product shown as reaction product in Eq. 1.

Because of the partial free-radical density on the phenolic oxygen, however, there is also a small extent of C-0 coupling, which reacts to give the biphenyl ether (2) which can be detected as an impurity in the reaction product at <3%.

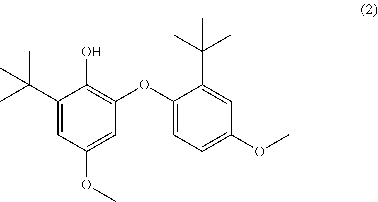 [SMILES: COc1ccc(Oc2cc(OC)cc(C(C)(C)C)c2O)c(C(C)(C)C)c1]

In one embodiment of the process according to the invention, the 3-tert-butyl-4-hydroxyanisole (3-BHA) reactant used is in an isomer mixture containing not only 3-tert-butyl-4-hydroxyanisole (3-BHA) but also 2-tert-butyl-4-hydroxyanisole (2-BHA). It has been found that the 2-tert-butyl-4-hydroxyanisole (2-BHA) isomer is essentially not converted, whereas the 3-tert-butyl-4-hydroxyanisole (3-BHA) isomer is converted quantitatively according to Eq. 1.

In one embodiment of the process according to the invention, the at least one solvent present in the mixture is selected from water, alcohols, hydrocarbons, amides. A particularly preferred solvent is water.

The inorganic base present in the mixture comprising the solvent and 3-tert-butyl-4-hydroxyanisole (3-BHA), in a preferred embodiment, is an alkali metal hydroxide, an alkaline earth metal hydroxide, an alkali metal carbonate or an alkaline earth metal carbonate, sodium hydroxide and potassium hydroxide being particularly preferred inorganic bases. Based on 1 equivalent of 3-BHA, preferably 1 to 3 equivalents, more preferably 1.5 to 2.5 equivalents and most preferably 1.8 to 2.2 equivalents of the inorganic base are used.

In one embodiment of the process, the mixture comprising the at least one solvent, the inorganic base and 3-tert-butyl-4-hydroxyanisole (3-BHA) additionally also comprises a surface-active compound. A preferred surface-active compound usable in the process according to the invention is sodium dodecylsulphate (sodium laurylsulphate). The surface-active compound can be used even in very small amounts, for example about 0.0001 to 0.2 equivalent based on 1 equivalent of 3-BHA. Preference is given to the use of 0.05 equivalent of surface-active substance based on 1 equivalent of 3-BHA.

According to the invention, the mixture comprising the at least one solvent, the inorganic base and 3-tert-butyl-4-hydroxyanisole (3-BHA) is heated to a temperature above room temperature. For example, the mixture is heated to a temperature between 30° C. and 100° C., especially to a temperature between 75° C. and 95° C. and more preferably to a temperature between 80° C. and 90° C.

In one embodiment, the heated mixture is stirred at elevated temperature for a duration of at least 15 minutes, for example at a temperature between 75° C. and 95° C., before the hydrogen peroxide solution is added. Preferably, the mixture is stirred at the elevated temperature for about 30 minutes prior to addition of the hydrogen peroxide solution.

The hydrogen peroxide solution which is added to the mixture comprising the at least one solvent, the inorganic base and 3-tert-butyl-4-hydroxyanisole (3-BHA) is preferably an aqueous hydrogen peroxide solution, for example a 30%-35% hydrogen peroxide solution. Preferably, for every equivalent of 3-BHA present in the mixture, one equivalent of the hydrogen peroxide solution is added to the mixture.

In one embodiment of the process, the hydrogen peroxide solution is added to the mixture at a temperature between 85° C. and 90° C. Optionally, after the addition of the hydrogen peroxide solution, stirring is effected at a temperature between 80° C. and 90° C. for a period of at least 15 minutes, especially for a period of about 30 minutes.

The 2,2′-dihydroxy-3,3′-di-tert-butyl-5,5′-dimethoxy-1,1′-biphenol product is preferably removed at room temperature. In one embodiment, the product can be removed from the solution by means of a centrifuge and/or a pressure suction filter.

Optionally, the product is subsequently dried, especially at elevated temperature and/or under reduced pressure, preferably until a water content of <0.1% is attained.

The product can thus be obtained with about an 85% yield (based on the total BHA content when an isomer mixture is used as reactant) and purities of >97%, the biphenyl ether 2 constituting the main impurity. Traces of monomeric 2-BHA and 3-BHA may likewise be present. Considering the reactive 3-BHA only, the yields obtained are actually quantitative.

The invention is to be illustrated in detail hereinafter by a working example.

### GENERAL PROCEDURES

The products were characterized by means of NMR spectroscopy. Chemical shifts (δ) are reported in ppm. The recording of nuclear resonance spectra was effected on Bruker Avance 300 or Bruker Avance 400, gas chromatography analysis on Agilent GC 7890A, elemental analysis on Leco TruSpec CHNS and Varian ICP-OES 715, and ESI-TOF mass spectrometry on Thermo Electron Finnigan MAT 95-XP and Agilent 6890 N/5973 instruments.

### Example 1: Preparation of 2,2′-Dihydroxy-3,3′-Di-Tert-Butyl-5,5′-Dimethoxy-1,1′-Biphenol

A flask is initially charged with 1360 ml of water. While stirring, 6 g of sodium n-dodecylsulphate, 320 g of sodium hydroxide and 721 g of 3-tert-butyl-4-hydroxyanisole (3-BHA) are added. The mixture is heated to 85° C. and left to stir at 85° C. for 30 minutes. Subsequently, 240 ml of hydrogen peroxide solution (35%) are added dropwise. During the addition of hydrogen peroxide solution, the temperature is kept between 85° C. and 90° C. After the addition has ended, the mixture is left to stir at 85° C. for a further 30 minutes. Then the mixture is allowed to cool down to room temperature, filtered and washed twice with 200 ml each time of water. The product is left to dry under reduced pressure.

Yield: 585.1 g (82%); purity 99%.

1. Process for preparing 2,2′-dihydroxy-3,3′-di-tert-butyl-5,5′-dimethoxy-1,1′-biphenol by oxidative coupling of 3-tert-butyl-4-hydroxyanisole, comprising the process steps of:
a) heating a mixture comprising at least one solvent, an inorganic base and 3-tert-butyl-4-hydroxyanisole to a temperature above room temperature,
b) adding a hydrogen peroxide solution to the mixture,
c) removing the 2,2′-dihydroxy-3,3′-di-tert-butyl-5,5′-dimethoxy-1,1′-biphenol product.

2. Process according to claim 1,
wherein the at least one solvent is selected from water, alcohols, hydrocarbons, amides.

3. Process according to claim 1,
wherein the inorganic base is an alkali metal hydroxide, an alkaline earth metal hydroxide, an alkali metal carbonate or an alkaline earth metal carbonate.

4. Process according to claim 3,
wherein the inorganic base is sodium hydroxide or potassium hydroxide.

5. Process according to claim 1,
wherein the mixture in step a) further comprises a surface-active compound.

6. Process according to claim 5,
wherein the surface-active compound is sodium dodecylsulphate.

7. Process according to claim 1,
wherein the mixture in step a) is heated to a temperature between 75° C. and 95° C.

8. Process according to claim 7,
wherein the mixture heated in step a) is stirred at the temperature between 75° C. and 95° C. for a period of at least 15 minutes before the hydrogen peroxide solution is added in step b).

9. Process according to claim 1,
wherein the addition of the hydrogen peroxide solution in step b) is effected at a temperature between 85° C. and 90° C.

10. Process according to claim 1,
wherein addition of the hydrogen peroxide solution in step b) is followed by stirring at a temperature between 80° C. and 90° C. for a duration of at least 15 minutes.

11. Process according to claim 1,
wherein the product is removed at room temperature.

12. Process according to claim 1,
wherein the removal of the product includes a treatment by means of a centrifuge and/or pressure suction filter.

13. Process according to claim 1,
wherein the 3-tert-butyl-4-hydroxyanisole is used as a constituent of an isomer mixture containing not only 3-tert-butyl-4-hydroxyanisole but also 2-tert-butyl-4-hydroxyanisole.
